(19) United States
(12) Patent Application Publication
(10) Pub. No.: US 2011/0226042 A1
(43) Pub. Date: Sep. 22, 2011

(54) CARBON DIOXIDE MEASURING DEVICE

(75) Inventors: Jung Kn Yu, Daejeon (KR); Jun Woong Kim, Daejeon (KR)

(73) Assignee: CIOS, Daejeon (KR)

(21) Appl. No.: 13/061,385

(22) PCT Filed: Dec. 4, 2009
(86) PCT No.: PCT/KR2009/007230

§ 371 Date: Feb. 28, 2011

(30) Foreign Application Priority Data
Dec. 5, 2008 (KR) 10-2008-0122925

(51) Int. Cl. G01N 33/00

(52) U.S. Cl. 73/31.05

(57) ABSTRACT

The present invention relates to a carbon dioxide measuring device, and more particularly, to a carbon dioxide measuring device in which a reference electrode signal transfer pattern is formed on one side of a heating unit to connect an electromotive lead wire for measuring carbon dioxide concentration to the outside of a portion at which the heating unit contacts a carbon dioxide sensor to thereby prevent generation of a gap, and a problem that a sealant is diffused into the reference electrode to have an effect on electromotive force is prevented to thereby increase durability and accuracy and also increase reliability of measurement of carbon dioxide concentration.

### TECHNICAL FIELD

The present invention relates to a carbon dioxide measuring device, and more particularly, to a carbon dioxide measuring device in which a reference electrode signal transfer pattern is formed on one side of a heating unit to connect an electromotive lead wire for measuring carbon dioxide concentration to the outside of a portion at which the heating unit contacts a carbon dioxide sensor to thereby prevent generation of a gap, and a problem that a sealant is diffused into the reference electrode to have an effect on electromotive force is prevented to thereby increase durability and accuracy and also increase reliability of measurement of carbon dioxide concentration.

### BACKGROUND ART

Carbon dioxide, which is a chemically very stable gas in the atmosphere, is a main cause of global warming. There is an increasing need to control carbon dioxide concentration for air-conditioning of building and gardening, in addition to an environmental problem. Therefore, research into a method of measuring the carbon dioxide gas concentration in the atmosphere has been actively conducted.

As a method of measuring the carbon dioxide gas concentration in the atmosphere, an optical method (an NDIR method) is currently mainly used. In this method, a degree of absorption is measured using the principle that carbon dioxide absorbs an infrared ray having a specific wavelength to thereby measure the carbon dioxide concentration.

This device is excellent in view of selectivity, quantitativeness, and reproducibility; however, it requires a closed space for measurement and is bulky and heavy due to a physical size of its components and filters. In addition, a driving unit and a measuring element are very expensive and a configuration of a processing unit for control is complicated, such that the entire measuring device is expensive. Therefore, although this device has various uses, it has not been widely utilized. Particularly, in the case in which this device is exposed to a poor environment, an optical system is easily polluted. Therefore, the usage thereof is limited to indoors.

As another method of measuring the carbon dioxide concentration, a semiconductor type gas sensor using a semiconductor compound such as SnO₂, TiO₂, or the like, may be used. The semiconductor type gas sensor measures the gas concentration through a resistance change generated when gas particles are adsorbed on a surface the semiconductor compound, such that it is manufactured to have a small size. However, since it is difficult to distinguish different kinds of adsorbed gas particles, gas selectivity is significantly low.

In order to solve the problem, a gas measuring device using a solid electrolyte has been suggested. The gas measuring device using a solid electrolyte has a simple structure, may be manufactured as a sensor having a small element form, and uses a sensing electrode selectively sensing specific gas, such that the gas selectivity is increased and the gas concentration may be quantitatively measured. In addition, the gas measuring device using a solid electrolyte is inexpensive, and uses an oxide, such that it may be stably used even under a severe environment.

The gas measuring device using a solid electrolyte has been continuously developed since the research into a carbon dioxide sensor using a potassium carbonate by Gauthier and Chamberland in 1970s. The research into an electromotive force type carbon dioxide sensor using a solid electrolyte such as NASICON, LISICON, β-alumina (NBA), etc., has been actively conducted for several years.

However, although the reason why an electrochemical carbon dioxide measuring device using a solid electrolyte has the above-mentioned many advantages, it has not been widely used due to the following main reason. It is necessary to maintain a constant high driving temperature of the electrochemical carbon dioxide measuring device using a solid electrolyte in order to smoothly operate the electrochemical carbon dioxide measuring device. However, a surrounding temperature may be rapidly changed, such that it is very difficult to maintain a constant driving temperature due to a large temperature difference between an inner temperature of a heater and the surrounding temperature. As a result, an output signal of the sensor is changed due to the temperature difference, such that the accuracy and the reliability of measurement of the carbon dioxide concentration are lowered.

More specifically, in the electrochemical carbon dioxide measuring device according to the related art, a galvanic cell (Type III) in which a mixed carbonate phase and a noble metal film generating thermodynamic equilibrium reaction with target gas to be sensed is used as a sensing electrode and a metal oxide/oxide mixed phase capable of constantly fixing the activity of Na₂O on an interface between an electrolyte and a reference electrode in a wide carbon dioxide concentration range and a wide temperature range without generating reaction with carbon dioxide is used as the reference electrode has been mainly used. A carbon dioxide sensing sensor having the galvanic cell structure is in accordance with the principle that electromotive force measured in electrodes positioned on both surfaces of an electrolyte is changed by Nernst equation according to surrounding carbon dioxide gas concentration, and shows a significantly stable measurement value (EMF) at a partial pressure of carbon dioxide gas at an appropriate driving temperature (350 to 550° C.).

However, in order to allow the sensor to be smoothly operated, ion conductivity of the solid electrolyte should be high and equilibrium reaction between the gas and the electrode should be maintained. Therefore, a heating unit is essential in the carbon dioxide measuring device.

Particularly, it is important to allow the heating unit to always constantly maintain a driving temperature of the carbon dioxide measuring device. When the driving temperature of the carbon dioxide measuring device is constant, the carbon dioxide measuring device generates a constant electromotive force value according to the carbon dioxide concentration, thereby making it possible to measure the carbon dioxide concentration.

However, when the driving temperature of the carbon dioxide measuring device is not constant, a signal change due to a temperature change rather than a signal change due to the concentration is generated, such that false information on target gas concentration to be measured may be provided, which hinders the carbon dioxide measuring device from being actually used.

In addition, the heating unit is degraded more rapidly than the carbon dioxide sensor due to deterioration, which serves as a main cause of the lowering of the overall durability.

Meanwhile, in the case in which a sealant is used in order to bond the carbon dioxide sensor to the heat unit in the carbon dioxide measuring device according to the related art, diffusion of the sealant into the reference electrode may occur during high temperature driving to thereby have an effect on a sensor signal, such that the reliability of the measurement of the carbon dioxide concentration is lowered.

### DISCLOSURE

### Technical Problem

An object of the present invention is to provide a carbon dioxide measuring device in which a signal of a reference electrode is transferred to one side of a heating unit to connect an electromotive lead wire to the outside of a contact portion between a carbon dioxide sensor and the heating unit, such that the electromotive lead wire may be provided between the carbon dioxide sensor and the heating unit, thereby making it possible to prevent a gap from being generated, and a diffusion preventing layer preventing a sealant from being diffused into the reference electrode is formed such that the carbon dioxide sensor and the heating unit may be uniformly bonded to each other, thereby making it possible to further increase reliability of measurement of carbon dioxide concentration.

In addition, an object of the present invention is to provide a carbon dioxide measuring device in which a protecting layer protecting a heat generating pattern of a heating unit is formed, such that influence from an external environment is minimized and deterioration of the heat generating pattern due to high temperature operation over a long time is suppressed, thereby making it possible to further increase stability of an output signal according to carbon dioxide concentration.

Further, an object of the present invention is to provide a carbon dioxide measuring device capable of having high durability, excellent reproducibility, and easiness in mass production to thereby increase productivity.

### Technical Solution

In one general aspect, there is provided a carbon dioxide measuring device including: a carbon dioxide sensor 100 including a solid electrolyte 110, and a reference electrode 120 and a sensing electrode 130 formed on one side or both sides of the solid electrolyte 110; a heating unit 200 including a ceramic substrate 210 having a heat generating part 220 formed on one side thereof; and an electromotive lead wire 300 extended to the reference electrode 120 and the sensing electrode 130.

The heating unit 200 may have at least one diffusion preventing layer 230 formed to enclose a circumference of the reference electrode 120 at a portion at which the heating unit 200 contacts the carbon dioxide sensor 100.

The diffusion preventing layer 230 may be made of a metal, an oxide or a mixture thereof, and more specifically, the diffusion preventing layer 230 may be made of at least one selected from a group consisting of Pt, Au, Ag, Al₂O₃, ZrO₂, and SiO₂.

A circumference at which the heating unit 200 contacts the carbon dioxide sensor 100 may be sealed by a sealant 400, and the sealant 400 may be formed to enclose the entire solid electrolyte 110 except for the sensing electrode 130 of the carbon dioxide sensor 100 and may be made of at least one selected from a group consisting of Al₂O₃, SiO₂, B₂O₃, PbO, BaO, CaO, and Si₃N₄.

The heating unit 200 may have a reference electrode signal transfer pattern 240 formed therein, the reference electrode signal transfer pattern 240 having a reference electrode contact 241 formed to contact the reference electrode 120 of the carbon dioxide sensor 100 on the other side of the ceramic substrate 210 on which the heat generating part 220 is not formed to thereby transfer a signal of the reference electrode 120, and the reference electrode signal transfer pattern 240 may include an extension part 242 extended from the reference electrode contact 241 to an edge portion of the ceramic substrate 210, such that the electromotive lead wire 300 is connected to the extension part 242 outside a bonding portion between the heating unit 200 and the carbon dioxide sensor 100.

The reference electrode signal transfer pattern 240 may be made of at least one selected from a group consisting of Pt, Au, Ru and Ag.

The heat generating part 220 may include: a heat generating pattern 221 formed on an upper side of the ceramic substrate 210; a pair of voltage application lead wires 222 connected to both ends of the heat generating pattern 221 to apply a voltage thereto; and a heat generating pattern protecting layer 223 formed on one side of the ceramic substrate 210 to include the heat generating pattern 221 therein, and the heat generating pattern protecting layer 223 may be made of at least one selected from a group consisting of Al₂O₃, SiO₂, Na₂O, Li₂O, B₂O₃, V₂O₅, MgO, ZnO, CaO, BaO, and Si₃N₄.

The sensing electrode 130 may include a mixed layer 131 formed on the solid electrolyte 110 and containing a noble metal and a carbonate and a porous noble metal protecting layer 132 formed to enclose the mixed layer 131, the porous noble metal protecting layer 132 having a noble metal as a main component, and the porous noble metal protecting layer 132 may be made of at least one selected from a group consisting of Au, Ag, Pt, SiO₂, B₂, P₂O₅, Na₂O, Li2O, and Al₂O₃.

### Advantageous Effects

With the carbon dioxide measuring device according to the exemplary embodiments of the present invention, the heating unit having the reference electrode signal transfer pattern formed therein is used, such the lead wire is formed outside the contact portion between the carbon dioxide sensor and the heating unit, thereby making it possible to more effectively bond the carbon dioxide sensor to the heating unit. In addition, the diffusion preventing layer capable of preventing the sealant from being diffused into the reference electrode is formed, thereby making it possible to further increase the reliability of the measurement of the carbon dioxide concentration.

Further, with the carbon dioxide measuring device according to the exemplary embodiments of the present invention, the heating unit including the heat generating pattern protecting layer protecting the heat generating pattern is used to prevent a shape of the heat generating pattern from being changed and minimize influence from the external environment, such that a constant driving temperature is provided, thereby making it possible to stably measure the carbon dioxide concentration.

Furthermore, the carbon dioxide measuring device according to the exemplary embodiments of the present invention has excellent reproducibility and easiness in mass production, thereby making it possible to increase productivity and durability.

### DESCRIPTION OF DRAWINGS

The above and other objects, features and advantages of the present invention will become apparent from the following description of preferred embodiments given in conjunction with the accompanying drawings, in which:

FIG. 1 is a cross-sectional view of a carbon dioxide measuring device according to the present invention;

FIG. 2 is a cross-sectional view of a carbon dioxide sensor of the carbon dioxide measuring device shown in FIG. 1;

FIG. 3 is a perspective view of a heating unit of the carbon dioxide measuring device shown in FIG. 1;

FIG. 4 is another cross-sectional view of a carbon dioxide measuring device according to the present invention;

FIG. 5 is a view showing a heating unit of the carbon dioxide measuring device shown in FIG. 4;

FIG. 6 is another cross-sectional view of a carbon dioxide measuring device according to the present invention;

FIG. 7A is a cross-sectional view of a carbon dioxide measuring device according to the related art and FIG. 7B is a graph showing electromotive force thereof according to a turn on/off test;

FIG. 8 is a graph showing electromotive forces of a carbon dioxide measuring device according to the related art and a carbon dioxide measuring device according to the present invention according to time;

FIG. 9 is a graph showing electromotive force of a carbon dioxide measuring device according to the present invention according to a turn on/off test; and

FIG. 10 is a graph showing electromotive forces of carbon dioxide measuring devices of each of Examples according to the present invention according to time.

[Detailed Description of Main Elements]

| | | | |
|---|---|---|---|
| 1000: | CARBON DIOXIDE | 100: | CARBON DIOXIDE |
| | MEASURING DEVICE | | SENSOR |
| 110: | SOLID ELECTROLYTE | 120: | REFERENCE |
| | | | ELECTRODE |
| 130: | SENSING ELECTRODE | 131: | MIXED LAYER |
| 132: | POROUS NOBLE | 200: | HEATING UNIT |
| | PROTECTING LAYER | | |
| 210: | CERAMIC SUBSTRATE | 220: | HEAT GENERATING |
| | | | PART |
| 221: | HEAT GENERATING | | |
| | PATTERN | | |
| 222: | VOLTAGE APPLICATION | | |
| | LEAD WIRE | | |
| 223: | HEAT GENERATING | | |
| | PATTERN PROTECTING | | |
| | LAYER | | |
| 230: | DIFFUSION PREVENTING | | |
| | LAYER | | |
| 240: | REFERENCE ELECTRODE | | |
| | SIGNAL TRANSFER | | |
| | PATTERN | | |
| 241: | REFERENCE ELECTRODE | 242: | EXTENSION PART |
| | CONTACT | | |
| 300: | ELECTROMOTIVE LEAD | 400: | SEALANT |
| | WIRE | | |

### BEST MODE

Hereinafter, the embodiments of the present invention will be described in detail with reference to accompanying drawings.

Hereinafter, a carbon dioxide measuring device 1000 according to the present invention having the above-mentioned characteristics will be described in detail with reference to the accompanying drawings.

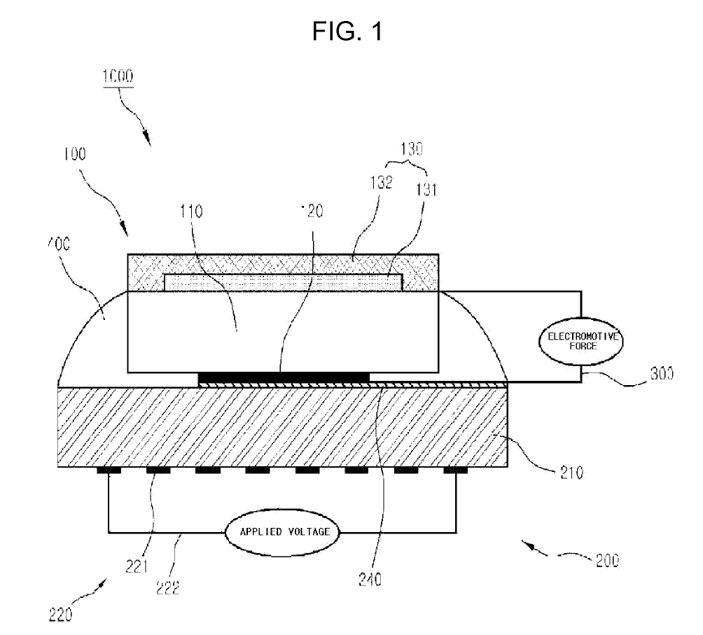

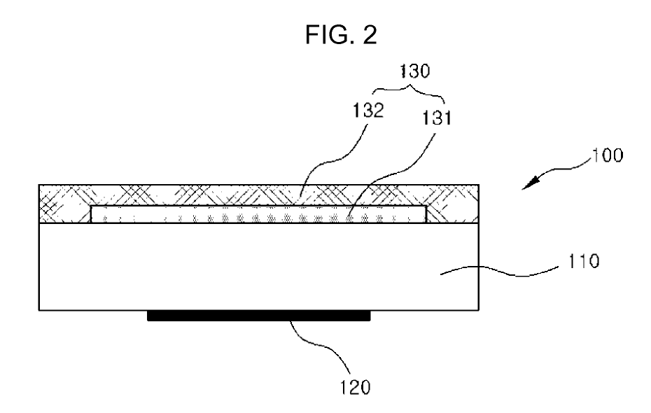

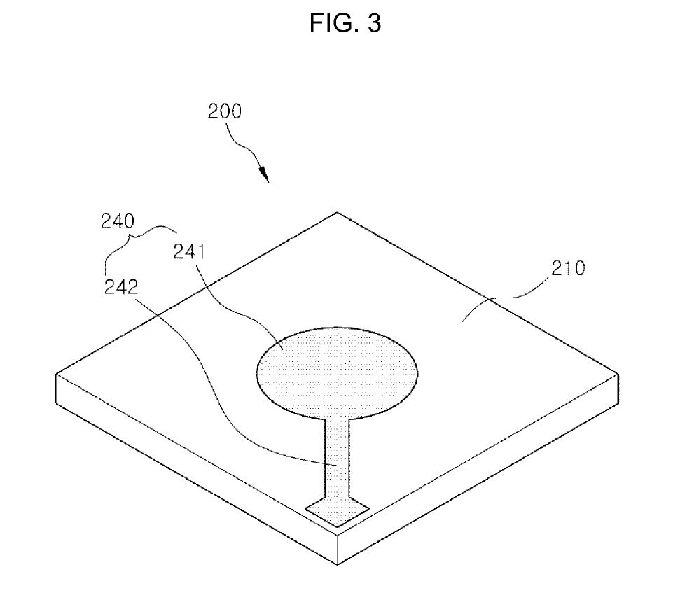

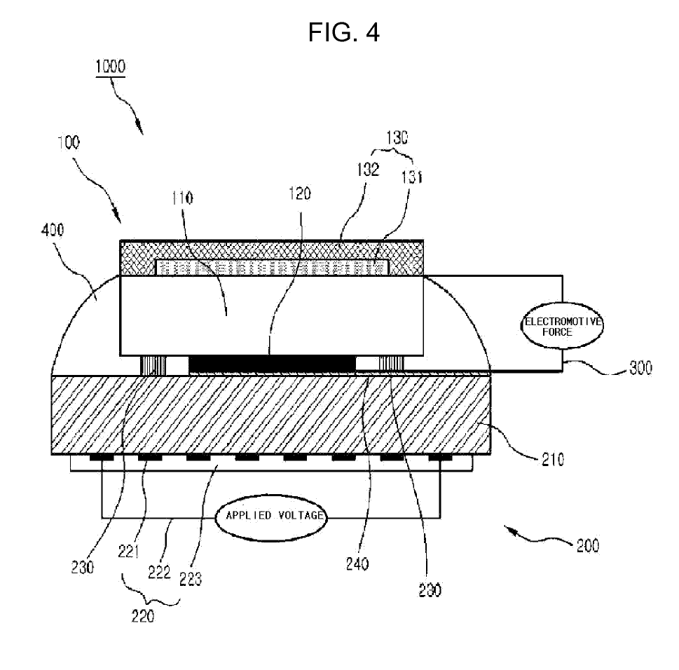

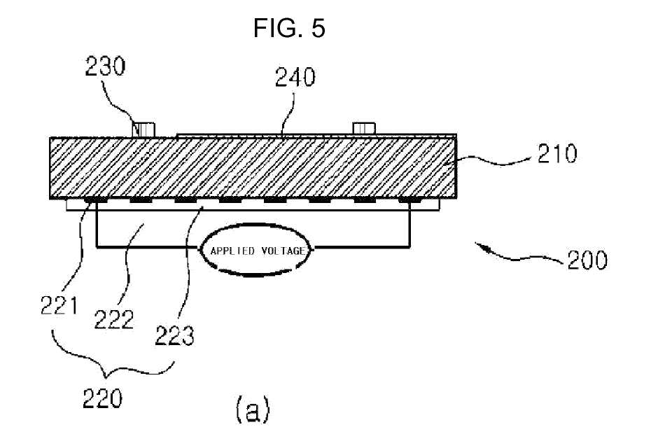

FIG. 1 is a cross-sectional view of a carbon dioxide measuring device 1000 according to the present invention; FIG. 2 is a cross-sectional view of a carbon dioxide sensor 100 of the carbon dioxide measuring device 1000 shown in FIG. 1; FIG. 3 is a perspective view of a heating unit 200 of the carbon dioxide measuring device 1000 shown in FIG. 1; FIG. 4 is another cross-sectional view of a carbon dioxide measuring device 1000 according to the present invention; and FIG. 5 is a view showing a heating unit 200 of the carbon dioxide measuring device 1000 shown in FIG. 4.

A carbon dioxide measuring device 1000 according to the present invention is mainly configured to include a carbon dioxide sensor 100, a heating unit 200 formed on one side of the carbon dioxide sensor 100 and heating the carbon dioxide sensor 100 to a constant temperature, and an electromotive lead wire 300 connected to the carbon dioxide sensor 100.

The carbon dioxide sensor 100 is in accordance with the principle that electromotive force measured in a sensing electrode 130 and a reference electrode 120 provided on both surfaces of a solid electrolyte 110 in a galvanic cell structure is changed by Nernst equation according to the surrounding carbon dioxide gas concentration.

Here, the solid electrolyte 110 is made of an alkali ion conductor such as lithium, sodium, potassium. More specifically, the solid electrolyte 110 may be made of β-alumina: Na₂O.χAl₂O₃, NASICON: Na_{1+y}Zr₂Si_{y}P_{3-y}O₁₂, an alkali metal carbonate such as Na₂CO₃, Li₂CO₃, K₂CO₃, Li₃PO₄, lithium phosphorous oxynitride (LIPON), LISICON(Li_{1+y}Zr₂Si_{y}P_{3-y}O₁₂), or a mixture thereof, wherein X is a constant of 1 to 11 and Y is a constant of 1 to 3.

In addition, the reference electrode 120 may be formed on one side of the solid electrolyte 110 bonded to the heating unit 200, and may be made of a noble metal, Na₂ZrO₃—ZrO₂, Na₂Mo0₄—MoO₃, Na₂WO₄—WO₃, Na₂SnO₃—SnO₂, Na₂Ti₆O₁₃—TiO₂, Na₂Ti₆O₁₃—Na₂Ti₃O₇, Na₂Si₂O₅—SiO₂, Na₂Si₂O₅—Na₂Si₁O₃, Na₂Ge₄O₃—GeO₂, Li₂TiO₃—TiO₂, LiCo0₂—Co₃O₄, or a mixture thereof, more preferably, Na₂Ti₆O₁₃—TiO₂, LiCo0₂—Co₃O₄, Li₂TiO₃—TiO₂, or a mixture thereof.

Further, the sensing electrode 130 preferably includes a mixed layer 131 of a noble metal and a carbonate formed on the solid electrolyte 110 and a porous noble metal protecting layer 132 formed to enclose the mixed layer 131.

More specifically, the mixed layer 131 of a noble metal and a carbonate preferably contains at least one carbonate selected from a group consisting of Na₂CO₃, BaCO₃, Li₂CO₃, SrCO₃, CaCO₃, Cs₂CO₃, MnCO₃, MgCO₃, K₂CO₃, Rb₂CO₃, and CuCO₃, and at least one noble metal selected from a group consisting of Pt, Au, and Ag, more preferably at least one carbonate selected from a group consisting of BaCO₃, Na₂CO₃, Li₂CO₃, and CaCO₃ and at least one noble metal selected from a group consisting of Pt and Au.

In addition, the porous noble metal protecting layer 132 may be made of a noble metal and an oxide, and may be made of at least one selected from a group consisting of Au, Ag, Pt, SiO₂, B₂O₃, P₂O₅, and Al₂O₃.

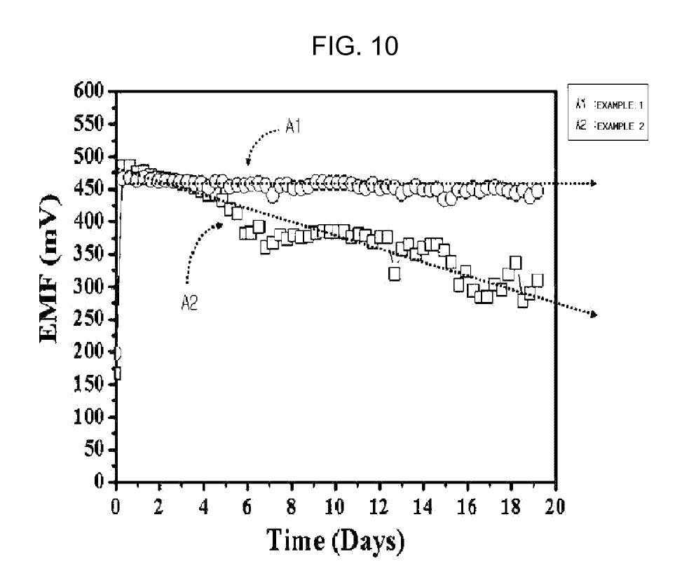

The carbon dioxide measuring device 1000 according to the present invention may include the sensing electrode 130 including the mixed layer 131 and the porous noble metal protecting layer 132. The sensing electrode 130 may suppress volatilization of the mixed layer 131 containing the carbonate even in a high temperature operation over a long time and prevent deterioration of an interface between the mixed layer 131 and the porous noble metal protecting layer 132 to increase durability, thereby making it possible to increase reliability of the carbon dioxide measuring device 1000, as compared to a case in which a carbonate layer 13a is formed on a porous noble layer 13b according to the related art, as shown in FIG. 10.

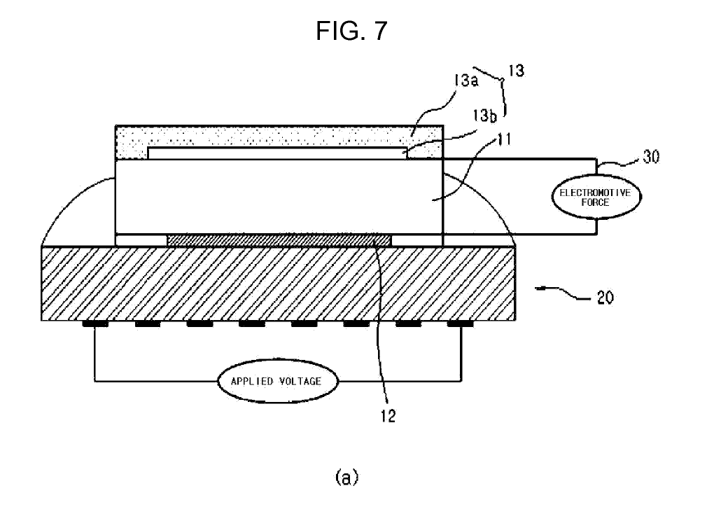

The carbon dioxide measuring devices of Example 1 and Example 2 of FIG. 10 have a structure shown in FIG. 4. The carbon dioxide measuring device of Example 1 in which the sensing electrode 130 includes the mixed layer 131 and the protecting layer 132 was compared with the carbon dioxide measuring device of Example 2 in which the sensing electrode 130 includes the noble layer 13b and the carbonate layer 13a according to the related art as shown in FIG. 7A. Example 1 and Example 2 will be again described in detail below.

The electromotive lead wire 300 measures carbon dioxide concentration by using electromotive force measured in both of the reference electrode 120 and the sensing electrode 130.

Here, since the carbon dioxide sensor 100 should have a constant driving temperature, it includes the heating unit 200 formed on one side thereof. In the carbon dioxide measuring device 1000 according to the present invention, the heating unit 200 is configured to include a ceramic substrate 210; a heat generating part 220 formed on one side of the ceramic substrate 210; and a reference electrode signal transfer pattern 240 having a reference electrode contact 241 formed to contact the reference electrode 120 of the carbon dioxide sensor 100 on the other side of the ceramic substrate 210 to thereby transfer a signal of the reference electrode 120.

That is, the heating unit 200 is formed with the heat generating part 220 for heating the carbon dioxide sensor 100 to an existing driving temperature and the reference electrode signal transfer pattern 240 for transferring the signal of the reference electrode 120.

The reference electrode signal transfer pattern 240 is configured to include the reference electrode contact 241 formed to contact the reference electrode 120 and an extension part 242 extended from the reference electrode contact 241 to an edge portion of the ceramic substrate 210.

The reference electrode signal transfer pattern 240 stably transfers the signal of the reference electrode 120, and the electromotive lead wire 300 capable of sensing the signal is connected to the outside of a bonding portion between the heating unit 200 and the carbon dioxide sensor 100 through the reference electrode signal transfer pattern 240, thereby making it possible to prevent a gap due to a thickness of the electromotive lead wire 300 from being formed between the heating unit 200 and the carbon dioxide sensor 100.

Therefore, in the carbon dioxide measuring device 1000 according to the present invention, the carbon dioxide sensor 100 and the heating unit 200 may be uniformly bonded to each other, damage due to heat or physical impact is reduced, thereby making it possible to increase durability, and defects due to non-uniform bonding is minimized, thereby making it possible to further increase productivity.

That is, the electromotive lead wire 300 is connected to the extension part 242 of the reference electrode signal transfer pattern 240 formed in the heating unit 200 and the sensing electrode 130.

Here, the reference electrode signal transfer pattern 240 may be preferably made of at least one selected from a group consisting of Pt, Au, Ru, and Ag, and may be formed by a method such as a screen printing method, a spray method, a doctor blade method, a spin coating method, a stencil printing method, etc.

In addition, in the carbon dioxide measuring device 1000 according to the present invention, the heat generating part 220 of the heating unit 200 is configured to include a heat generating pattern 221; a pair of voltage application lead wires 222 connected to both ends of the heat generating pattern 221 to apply a voltage thereto; and a heat generating pattern protecting layer 223 protecting the heat generating pattern 221 (See FIG. 5C).

The heat generating pattern 221 is formed on the ceramic substrate 210 to heat the entire region. The heat generating pattern 221 may be formed by applying a method such as a screen printing method, a spray method, a doctor blade method, a spin coating method, a stencil printing method, etc., to a material such as Pt paste. In addition to the above-mentioned material and method, various materials and methods may be used to form the heat generating pattern.

The heat generating pattern protecting layer 223 is formed on one side of the ceramic substrate 210 on which the heat generating pattern 221 is formed and includes the heat generating pattern 221 therein. The carbon dioxide measuring device 1000 according to the present invention has the heat generating pattern protecting layer 223 formed therein to suppress a resistance change, a shape change, or the like, of the heat generating pattern 221 due to an external environment, thereby making it possible to maintain a constant high driving temperature required for measuring the carbon dioxide concentration regardless of the external environment.

The heat generating pattern protecting layer 223 is preferably made of at least one selected from a group consisting of Al₂O₃, SiO₂, Na₂O, Li₂O, B₂O₃, V₂O₅, MgO, ZnO, CaO, BaO, and Si₃N₄.

A bonding circumference between the carbon dioxide sensor 100 and the heating unit 200 is bonded by a sealant 400, which is preferably made of at least one selected from a group consisting of Al₂O₃, SiO₂, B₂O₃, PbO, BaO, CaO, and Si₃N₄.

The sealant 400 is formed along circumferential surfaces of the carbon dioxide sensor 100 and the heating unit 200 to serve to prevent foreign materials from being penetrated from the outside, while bonding the carbon dioxide sensor 100 to the heating unit 200.

However, when the sealant 400 is diffused into the reference electrode 120, a change is caused in measured electromotive force. Therefore, the carbon dioxide measuring device 1000 according to the present invention preferably further includes a diffusion preventing layer 230 formed at a portion at which the heating unit 200 contacts the carbon dioxide sensor 100, the diffusion preventing layer 230 enclosing a circumference of the reference electrode 120 (See FIG. 5B).

The diffusion preventing layer 230 may be made of a metal, an oxide, or a mixture thereof. More specifically, the diffusion preventing layer 230 may be preferably made of at least one selected from a group consisting of Pt, Au, Ag, Al₂O₃, ZrO₂ and SiO₂, and may be formed by a method such as a screen printing method, a spray method, a doctor blade method, a spin coating method, a stencil printing method, etc.

Accordingly, the carbon dioxide measuring device 1000 according to the present invention may prevent the sealant 400 used for bonding between the carbon dioxide sensor 100 and the heating unit 200 from being diffused into the reference electrode 120, and allow an electromotive force signal to be constant, thereby making it possible to stably measure the carbon dioxide concentration.

In the carbon dioxide measuring device 1000 according to the present invention, the diffusion preventing layer 230 is formed in plural, thereby making it possible to more surely block the diffusion of the sealant 400.

An example in which the reference electrode signal transfer pattern 240 is formed together with the diffusion preventing layer 230 is shown in FIGS. 4 and 5. When the reference electrode signal transfer pattern 240 and the diffusion preventing layer 230 are made of the same material, they may also be formed by only a single process.

The diffusion preventing layer 230 is formed at a portion at which the heating unit 200 contacts the carbon dioxide sensor 100 on one side of the heating unit 200. In addition, the diffusion preventing layer 230 is formed to enclose the circumference of the reference electrode 120, thereby preventing external materials including the sealant 400 from being diffused into the reference electrode 120.

An example in which the reference electrode signal transfer pattern 240 is first formed and the diffusion preventing layer 230 is formed is shown in FIGS. 4 and 5.

Here, a portion at which the reference electrode signal transfer pattern 240 and the diffusion preventing layer 230 are overlapped with each other is preferably formed to have the same height as that of a portion at which only the diffusion preventing layer 230 is formed, such that the diffusion preventing layer 230 surely encloses the reference electrode 120, thereby making it possible to increase a sealing effect.

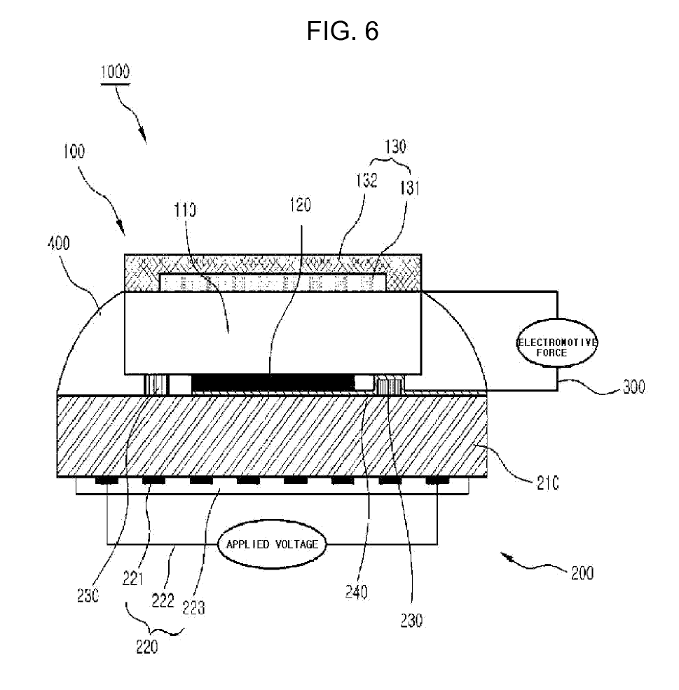

FIG. 6 is another cross-sectional view of a carbon dioxide measuring device 1000 according to the present invention, wherein an example in which the diffusion preventing layer 230 is first formed and the reference electrode signal transfer pattern 240 is formed is shown. A predetermined region of the reference electrode signal transfer pattern 240 prevents the external materials including the sealant 400 from being diffused into the reference electrode 120, together with the diffusion preventing layer 230.

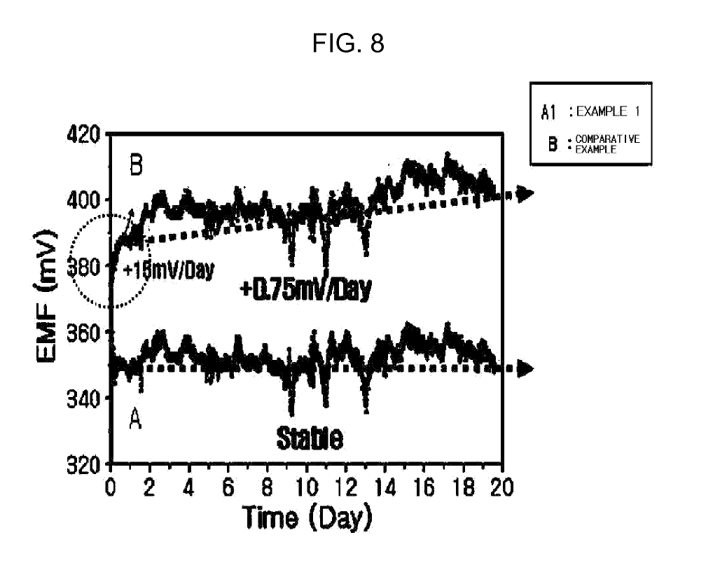

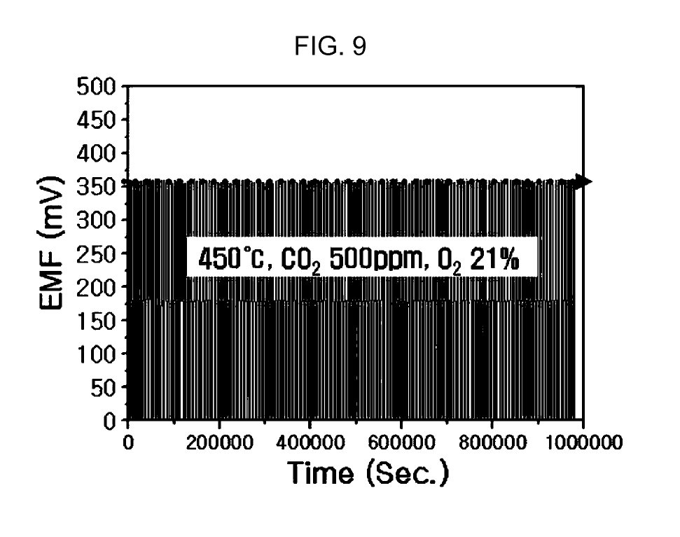

FIG. 7A is a cross-sectional view of a carbon dioxide measuring device 1000 according to the related art and FIG. 7B is a graph showing electromotive force thereof according to a turn on/off test; FIG. 8 is a graph showing electromotive forces of a carbon dioxide measuring device 1000 according to the related art and a carbon dioxide measuring device 1000 according to the present invention according to time; FIG. 9 is a graph showing electromotive force of a carbon dioxide measuring device 1000 according to the present invention according to a turn on/off test.

### Example 1

Example 1 of the present invention described the carbon dioxide measuring device 1000 having a structure shown in FIG. 4. First, in order to synthesize an NBA (Na₂.11Al₂O₃) solid electrolyte 110, Na₂CO₃ (Aldrich Co.) and Al₂O₃ (Aldrich Co.) powders were weighed in a molar ratio of 1:11 and were then wet ball-milled together with zirconia balls. The ball-milled mixture was dried and grinded in an oven for 12 hours and was then calcined at 1450° C. for 24 hours. Powders obtained through the above-mentioned process was molded to have a pellet shape, a green body of a dense NBA solid electrolyte 110 pellet was manufactured under cold isostatic pressure (CIP) of 250 Mpa and was then sintered at 1650° C. for 3 hours in air to thereby form an NBA solid electrolyte 110 pellet.

A Na₂Ti₆O₁₃—TiO₂ mixture, which is a material of the reference electrode 120, was prepared by weighing Na₂CO₃(Aldrich Co.) and TiO₂(Aldrich Co.) powders in a molar ratio of 1:6, wet ball-mill them together with zirconia balls, drying and grinding the ball-milled mixture in an oven for 12 hours and then, performing heat treatment at 1050° C. for 24 hours. Then, the reference electrode 120 was manufactured on one side of the NBA solid electrolyte 110 pellet formed through sintering by mixing the Na₂Ti₆O₁₃—TiO₂ mixture prepared as described above and Pt (Heraus Co.) in a volume ratio of 1:1 to form a paste, forming an electrode having a thickness of about 40 μm and a predetermined area using a screen printing method and performing heat treatment at 1000° C. for 30 minutes.

In addition, the heating unit 200 was manufactured on one surface of an alumina substrate through the following process. Pt (Heraus Co.) was first prepared in a paste form, the heat generating pattern 221 representing a predetermined resistance was formed using the screen printing method, the voltage application lead wire 222 was bonded to an end of the heat generating pattern 221 and was then introduced into the oven to be subjected to heat treatment at 1000° C. in the oven for 30 minutes. The heat generating pattern protecting layer 223 was applied over an upper portion of the heat-treated heat generating pattern 221 and was heat treated at 1000° C. by the same method as that of the heat generating pattern 221.

Then, the reference electrode signal transfer pattern 240 for connecting an electrical signal of the reference electrode 120 of the sensor 100 was manufactured on a side opposite to the heating generating pattern 221 by preparing Pt (Heraus Co.) in a paste form, performing the screen printing as described above, and then performing heat treatment at 950° C. The diffusion preventing layer 230 was similarly formed on the reference electrode signal transfer pattern 240 formed as described above by screen printing a Pt (Heraus Co.) paste and was then disposed on the reference electrode 120 of the sensor 100 manufactured as described above so as to contact thereto. At the same time, the electromotive lead wire 300 was bonded to the end of the reference electrode signal transfer pattern 240 and heat treatment was performed at 950° C.

In order to bond the heat treated sensor 100 to the heating unit 200, the sealant 400 was formed along the circumferences of the sensor 100 and the heating unit 200 to enclose the entire side of the solid electrolyte 110 of the sensor 100 and was subjected to the heat treatment at 950° C.

In order to process the sensing electrode 130 of the sensor manufactured as described above, a carbonate mixture in which Na₂CO₃(Aldrich Co.) and BaCO₃(Aldrich Co.) are mixed in a molar ratio of 1:1.7 and Au (Heraus Co.) was mixed in a volume ratio of 1:1 to prepare a paste and the paste was screen printed to form the mixed layer 131 having a thickness of 40 μm on another surface of the NBA solid electrolyte pellet, the lead wire was manufactured by bonding a Pt wire, and heat treatment was performed at 740° C. for 12 minutes. Then, the porous noble metal protecting layer 132 was manufactured by coating a paste containing a noble metal material Au (Heraus Co.) at a thickness of 40 μm similarly using the screen printing method and performing heat treatment at 670° C. for 12 minutes to thereby form the sensing electrode 130, such that the carbon dioxide measuring device 1000 is completed.

FIGS. 8 and 9 each shows a graph of the electromotive force of the carbon dioxide measuring device 1000 manufactured by Example 1 in the general office atmosphere according to time, while being maintained at a driving temperature of 450° C., and a graph of the electromotive force thereof according to a repetitive turn on/off test under a constant carbon dioxide concentration of 500 ppm.

FIG. 7A is a cross-sectional view of a carbon dioxide measuring device according to the related art; and FIG. 7B shows a graph of the electromotive force of the carbon dioxide measuring device of Comparative Example according to a repetitive turn on/off test under a constant carbon dioxide concentration of 500 ppm, in which the carbon dioxide measuring device has a structure shown in FIG. 7A and components thereof overlapped with the carbon dioxide measuring device 1000 according to the present invention are made of the same material.

A carbon dioxide measuring device according to the related art shown in FIG. 7A is configured to include a carbon dioxide sensor including a solid electrolyte 11 and a reference electrode 12 and a sensing electrode 13 formed on each of both sides of the solid electrolyte 11; a heating unit 20 bonded to a side on which the reference electrode 12 of the carbon dioxide sensor is positioned by a sealant and heating the carbon dioxide sensor; and an electromotive lead wire 30 connected to the reference electrode 12 and the sensing electrode 13, wherein the sensing electrode 13 includes a porous noble metal layer 13b and a carbonate layer 13a formed to include the porous noble metal layer 13b therein.

As shown in FIGS. 8 and 9, it may be confirmed that a signal value is not gradually changed and a constant electromotive force value is measured in the case of the carbon dioxide measuring device 1000 according to the present invention, thereby making it possible to stably measure the carbon dioxide concentration, as compared to Comparative Example.

More specifically, it may be confirmed that while the electromotive force signal is constant and is then rapidly broken down according to the repetitive turn on/off in the case of Comparative Example as shown in FIG. 7B, the electromotive force value is constant even by a continuous turn on/off test in the case of the carbon dioxide measuring device 1000 according to the present invention as shown in FIG. 9.

Further, in FIG. 8, in the case of Comparative Example, the signal value is gradually increased as time passes, such that the electromotive force value measured according to Comparative Example may not be used as accurate measurement data of the carbon dioxide concentration.

More specifically, it may be confirmed that the electromotive force is increased by about 15 mV for initially one day and is increased at a speed of 0.75 mV/Day as a review result of subsequent long-term stability, in the case of Comparative Example.

It may be confirmed that the electromotive force value is more stably measured in the case of the carbon dioxide measuring device 1000 according to the present invention, thereby making it possible to increase reliability of the measurement of the carbon dioxide concentration.

### Example 2

The carbon dioxide measuring device of Example 2 has the same structure as that of the carbon dioxide measuring device of Example 1 and is manufactured using the same material as that of the carbon dioxide measuring device of Example 1. However, the sensing electrode 130 includes the noble metal layer 13b and the carbonate layer 13a according to the related art, as shown in FIG. 7A.

More specifically, in the sensing electrode 130 of Example 2, a paste containing Au (Heraus Co.) was coated at a thickness of about 40 μm on another surface of the NBA solid electrolyte pellet using the screen printing method, the Pt wire was bonded to manufacture the lead wire, heat treatment was then performed at 670° C. for 12 minutes to form the noble metal layer.

Then, a carbonate in which Na₂CO₃ (Aldrich Co.) and BaCO₃(Aldrich Co.) are mixed in a molar ratio of 1:1.7 was prepared similarly in a paste form, was screen printed to form a carbonate layer having a thickness of 40 μm, and heat treatment was performed at 740° C. for 12 minutes.

That is, the carbon dioxide measuring device according to Example 2 includes the sensing electrode 130 having a structure according to the related art; however, it also includes the diffusion preventing layer 230 and the reference electrode signal transfer pattern 240 formed in the heating unit 200 contacting the reference electrode, which is another feature of the present invention, as shown in FIG. 4. FIG. 10 shows a graph of electromotive forces of carbon dioxide measuring devices of each of Examples according to the present invention according to time.

As seen in FIG. 10, in the carbon dioxide measuring device 1000 according to the present invention, the sensing electrode 130 includes the mixed layer 131 and the porous noble metal protecting layer 132 as well as the heating unit 200 includes the diffusion preventing layer 230 and the reference electrode signal transfer pattern 240 formed therein, thereby making it possible to stably measure the carbon dioxide concentration.

That is, in the carbon dioxide measuring device 1000 according to the present invention, bonding performance is increased, thereby making it possible to increase durability, a problem due to the diffusion of the sealant 400, etc., is prevented in advance, and the volatilization of the mixed layer 131 of the sensing electrode 130 is suppressed, thereby making it possible to further increase reliability.

The present invention is not limited to the above-mentioned exemplary embodiments, and may be variously applied, and may be variously modified without departing from the gist of the present invention claimed in the claims.

1. A carbon dioxide measuring device comprising:
a carbon dioxide sensor 100 including a solid electrolyte 110, and a reference electrode 120 and a sensing electrode 130 formed on one side or both sides of the solid electrolyte 110;
a heating unit 200 including a ceramic substrate 210 having a heat generating part 220 formed on one side thereof; and
an electromotive lead wire 300 extended to the reference electrode 120 and the sensing electrode 130.

2. The carbon dioxide measuring device according to claim 1, wherein the heating unit 200 has at least one diffusion preventing layer 230 formed to enclose a circumference of the reference electrode 120 at a portion at which the heating unit 200 contacts the carbon dioxide sensor 100.

3. The carbon dioxide measuring device according to claim 2, wherein the diffusion preventing layer 230 is made of a metal, an oxide or a mixture thereof.

4. The carbon dioxide measuring device according to claim 3, wherein the diffusion preventing layer 230 is made of at least one selected from a group consisting of Pt, Au, Ag, Al₂O₃, ZrO₂, and SiO₂.

5. The carbon dioxide measuring device according to claim 2, wherein a circumference at which the heating unit 200 contacts the carbon dioxide sensor 100 is sealed by a sealant 400, and the sealant 400 is formed to enclose the entire solid electrolyte 110 except for the sensing electrode 130 of the carbon dioxide sensor 100.

6. The carbon dioxide measuring device according to claim 5, wherein the sealant 400 is made of at least one selected from a group consisting of Al₂O₃, SiO₂, B₂O₃, PbO, BaO, CaO, and Si₃N₄.

7. The carbon dioxide measuring device according to any one of claims 1 to 6, wherein the heating unit 200 has a reference electrode signal transfer pattern 240 formed therein, the reference electrode signal transfer pattern 240 having a reference electrode contact 241 formed to contact the reference electrode 120 of the carbon dioxide sensor 100 on the other side of the ceramic substrate 210 on which the heat generating part 220 is not formed to thereby transfer a signal of the reference electrode 120.

8. The carbon dioxide measuring device according to claim 7, wherein the reference electrode signal transfer pattern 240 includes an extension part 242 extended from the reference electrode contact 241 to an edge portion of the ceramic substrate 210, such that the electromotive lead wire 300 is connected to the extension part 242 outside a bonding portion between the heating unit 200 and the carbon dioxide sensor 100.

9. The carbon dioxide measuring device according to claim 8, wherein the reference electrode signal transfer pattern 240 is made of at least one selected from a group consisting of Pt, Au, Ru and Ag.

10. The carbon dioxide measuring device according to claim 1, wherein the heat generating part 220 includes: a heat generating pattern 221 formed on one side of the ceramic substrate 210; a pair of voltage application lead wires 222 connected to both ends of the heat generating pattern 221 to apply a voltage thereto; and a heat generating pattern protecting layer 223 formed on an upper side of the ceramic substrate 210 to include the heat generating pattern 221 therein.

11. The carbon dioxide measuring device according to claim 10, wherein the heat generating pattern protecting layer 223 is made of at least one selected from a group consisting of Al₂O₃, SiO₂, Na₂O, Li₂O, B₂O₃, V₂O₅, MgO, ZnO, CaO, BaO, and Si₃N₄.

12. The carbon dioxide measuring device according to claim 1, wherein the sensing electrode 130 includes a mixed layer 131 formed on the solid electrolyte 110 and containing a noble metal and a carbonate, and a porous noble metal protecting layer 132 formed to enclose the mixed layer 131, the porous noble metal protecting layer 132 having a noble metal as a main component.

13. The carbon dioxide measuring device according to claim 12, wherein the porous noble metal protecting layer 132 is made of at least one selected from a group consisting of Au, Ag, Pt, SiO₂, B₂O₃, P₂O₅, Na₂O, Li₂O, and Al₂O₃.
